(19) United States
(12) Patent Application Publication
(10) Pub. No.: US 2020/0061130 A1
(43) Pub. Date: Feb. 27, 2020

(54) COMPOSITIONS AND METHODS OF TREATMENT OF CHRONIC INFECTIOUS DISEASES

(71) Applicant: Thomas Julius BORODY, Five Dock (AU)

(72) Inventor: Thomas Julius BORODY, Five Dock (AU)

(21) Appl. No.: 16/079,356

(22) PCT Filed: Feb. 24, 2017
(86) PCT No.: PCT/AU2017/000055

§ 371 Date: Aug. 23, 2018

Related U.S. Application Data
(60) Provisional application No. 62/299,915, filed on Feb. 25, 2016.

(51) Int. Cl. A61K 35/741; A61K 35/37; A61P 1/00; A61K 45/06; A61P 31/04

(52) U.S. Cl. CPC A61K 35/741; A61K 35/37; A61K 2035/115; A61K 45/06; A61P 31/04; A61P 1/00

(57) ABSTRACT

In alternative embodiments, provided are novel applications of bacteria which originate from the phylum of Actinobacteria and sub-order Corynebacterineae, family Dietziaceae, including genus Dietzia and other genera. Such bacilli can profoundly interfere with bacteria generally belonging to this and other phyla, and can be useful in treating chronic infections. Hence, such organisms can ameliorate or cure clinical infections caused by pathogens from this phylum such as Mycobacteriaceae and Mycobacterium such as M. tuberculosis and Mycobacterium avium sub species paratuberculosis (MAP).

### CROSS-REFERENCE TO RELATED APPLICATIONS

This application claims priority from patent application U.S. Ser. No. 62/299,915, filed 25 Feb. 2016, the contents of which are incorporated herein in entirety.

### TECHNICAL FIELD

The present disclosure relates to pharmaceutical compositions for the treatment in mammals of chronic conditions frequently associated with infective agents. In particular, provided herein are pharmaceutical compositions and methods of treatment of infections mediated by acid fast bacilli and other mycobacteria-like agents in humans and non-human mammals. Provided are novel applications of bacteria which originate from the phylum of Actinobacteria and sub-order Corynebacterineae, family Dietziaceae, including genus Dietzia and other genera. Such bacilli can profoundly interfere with bacteria generally belonging to this and other phyla, and can be useful in treating chronic infections. Hence, pharmaceutical compositions and methods as providing herein using such organisms can ameliorate or cure clinical infections caused by pathogens from this phylum such as Mycobacteriaceae and Mycobacterium such as M. tuberculosis and Mycobacterium avium subspecies paratuberculosis (MAP).

### BACKGROUND

There are a number of classes, orders and sub-orders, families, genera and species in the phylum Actinobacteria, but perhaps the most relevant pathogenic group are the Mycobacteriaceae. Within that, the genus of Mycobacterium stands out in that there are a number of species which are known for their pathogenicity. The best known genus is Mycobacterium tuberculosis which is the causative agent of most cases of tuberculosis. Within this genus there are a number of species which include Mycobacterium africanum, Mycobacterium bovis which one can acquire from drinking unpasteurised milk, Mycobacterium bovis BCG, Mycobacterium caprae, Mycobacterium microti, Mycobacterium mungi, Mycobacterium orygis, Mycobacterium suricattae and Mycobacterium pinnipedii. There are other lesser subgroups including M. canettii and Mycobacterium prototuberculosis.

Apart from the Mycobacterium tuberculosis group, there is another large group of mycobacteria called atypical mycobacteria. The most common atypical mycobacteria that cause disease are Mycobacterium avium complex (MAC). Others can cause localised disease such as Mycobacterium fortuitum complex, and Mycobacterium kansasii. Atypical mycobacteria are less aggressive than Mycobacterium tuberculosis but can nevertheless cause longstanding relapsing disease, for example in the lung. Atypical mycobacteria in particular, become more aggressive in patients with Acquired Immune Deficiency Syndrome (AIDS). Such atypical mycobacteria can cause many types of infections including pneumonia, lung abscess, pleural space infection, lymph node inflammation, skin and soft tissue infection, meningitis, gastrointestinal infection such as Crohn's disease, joint space infection, osteomyelitis, disseminated infection and even intravenous catheter related infections. More than 100 species of atypical mycobacteria have been described and many have been implicated in human infection. Established pathogens include M. avium intracellulare complex which may include M. avium avium, M. avium sylvaticum, and M. avium paratuberculosis, mostly associated with Crohn's disease and sarcoidosis. Also M. avium “hominssuis”, M. colombiense and M. indicus pranii. Other established pathogens include M. haemophilum; M. kansasii; M. leprae—the mycobacterium responsible for causing leprosy; M. malmoense; M. marinum; M. scrofulaceum; M. simiae; M. szulgai; M. ulcerans; M. gordonae; and M, xenopi. Other rapidly growing mycobacteria include M. abscessus; M. chelonae; and M. fortuitum. This is not an exhaustive list but it illustrates the diversity of both typical mycobacteria and atypical mycobacteria.

Mycobacterium avium subspecies paratuberculosis was first described in 1895 in Heidelberg by Johne and Frothingham. They noted that cattle developed a wasting disorder with weight loss and diarrhoea in the latter stages of the illness, and were infected with Mycobacterium avium subspecies paratuberculosis which caused chronic inflammation of the bowel in these animals and progressive weight loss and death ultimately. It affects both cows and sheep in Europe, USA and Australia. Indeed Johne's disease is of quite serious proportions worldwide and has a $1-2 billion economic impact due to reduced milk production with a loss income to farmers, and need for increased culling of animals, low weights and extended calving intervals. There is no effective treatment for paratuberculosis infection in sheep and cattle.

It has been postulated that Crohn's disease, which in many ways resembles that of Johne's disease, could be mediated by infection with Mycobacterium-like pathogens. Indeed, such agents have been identified from time to time but have been very difficult to culture in the past. The treatment with antibiotics of Mycobacterium tuberculosis requires combination treatments with multiple antibiotics for a prolonged period of time—many months. Atypical mycobacteria are classically resistant to antibiotics and although a proportion of Mycobacterium avium avium infection in humans without an immunosuppression can be cured, a large number of patients have to maintain antibiotic therapy otherwise their lungs will be progressively destroyed. The major mycobacterial human infections are Mycobacterium tuberculosis which has killed about 1 billion people over the past two centuries and Mycobacterium leprae which afflicts around 10 million people worldwide. Mycobacteria are also thought to be the underlying cause of sarcoidosis, and Crohn's disease which afflicts at least 5 million people worldwide at any one time, and is considered to be caused by Mycobacterium avium subspecies paratuberculosis-like organisms.

Because of the slow division of Mycobacterium avium subspecies paratuberculosis (MAP) in culture requiring at times up to one year to grow the bacteria, it is difficult to work with to determine it's presence and susceptibility to antibiotics. Also, there is a need for effective treatments for all mycobacterial-induced disorders but particularly those like Crohn's disease which affects young people and who may require numerous operations e.g. hemi-colectomies and total colectomies ultimately living much of their life with a stoma in place. Crohn's disease in man is a chronic debilitating disorder characterised by chronic inflammation of the small and large bowel which can stricture, form fistulae, cause anemia, weight loss and diarrhoea. Over 20,000 patients are newly diagnosed each year within the USA alone and the disease remains with the patient suborder for life. It typically is seen as a granulomatous ileocolitis which causes deep ulceration and stricturing in the ileum when diagnosed colonoscopically. In publications on Crohn's disease, there is little discussion of the exact etiology and it still thought to be an “inappropriate and ongoing activation of the mucosal immune system driven by the presence of normal luminal flora” in genetically predisposed people (Podoslky D K. Inflammatory Bowel Disease. N Eng J Med 2002; 347:417-429).

Perhaps due to the fact that until now we have not been able to readily detect MAP in many patients with Crohn's disease, and because there is no effective curative therapy, or even a therapy that produces profound suppression of inflammation, there has been little uptake by gastroenterologists worldwide of the MAP hypothesis, and most continue to treat the effects of the infection i.e. inflammation, with various anti-inflammatory medications such as azulfidine, mesalasine, steroids, azathioprine, 6-mercaptopurine, methotrexate, infliximab or adalimumab among others. However, there is good evidence that MAP is transmitted through milk because it has been detected in milk cartons in food stores, through water, and in meat. Thus, what has been needed was a simple detection of MAP in patients with Crohn's disease as a model of atypical mycobacterial infection for this invention—and also an effective therapy that can treat and cures the MAP infection. This infection almost exclusively resides intracellularly in humans where it takes the form of a ‘cell wall deficient’ L-form bacterium, making it more difficult to stain with the classic TB stain—Ziehl Neelsen stain. In man it is also present in very low numbers, being termed a ‘paucimicrobial’ disease. On the other hand, in cattle with Johne's disease the bacterium has a cell wall which takes up this stain and is easily found in large numbers during the latter stages of the disease, so being called a ‘pluribacillary’ infection. The clinical presentation of Crohn's disease can be mimicked by other infections. Hence it is perhaps more useful to call this condition ‘Crohn's Syndrome’ as a virtually identical condition can result from other infections including M. tuberculosis, Entamoeba histolytica, Yersinia ssp and others (Campbell J et al Open J Int Med 2012;2:107). Treatment with combinations of antibiotics can control and arrest the MAP infection and place the Crohn's inflammation into remission healing the bowel. But this requires the use of high doses of antibiotics for many months to years, and upon ceasing the treatment the MAP can regrow and the disease restart.

Clostridium difficile can be inhibited by non-pathogenic C. difficile bacteria. In fact, there is now, under development by Seres Therapeutics, a mix of Clostridium spores which are of non-pathogenic C. difficile strains and when ingested in a capsule, they can eradicate relapsing C. difficile infection in close to 90% of patients.

R. Click has previously described the use of one such strain of Dietzia in inhibiting Mycobacteria infecting cows with Johne's disease (see U.S. Pat. No. 8,231,867 and U.S. Pat. No. 8,414,886). In these granted Patents, Dietzia deposited with the American type culture collection as accession number PTA-4125 was described as being able to inhibit Mycobacterium avium ssp paratuberculosis. It was specifically describing one particular strain also known as Dietzia species C79793-74. In his disclosure, Click had chosen a single strain of Dietzia and demonstrated in numerous experiments that when given early to cattle with low numbers of infective MAP, the orally administered Dietzia was capable of inhibiting their growth and in fact eradicating the infection in about one third of the cattle. However, MAP in cattle and in sheep is not the same genetic strain as it is in humans. In fact there are numerous MAP strains. Humans may have ‘humanised forms’ of cattle MAP or humanised forms of sheep MAP and other various MAP as it is present in many feral animals and also in deer and has been found in dogs. So similarly in humans there are numerous closely related yet different genetic strains of MAP organisms.

Because the Dietzia is capable of penetrating the intracellular environment where MAP resides in Crohn's disease and other conditions caused by Mycobacteria, in effect Dietzia may be functioning as a type of an intra-cellular ‘antibiotic’. Some of these acid fast bacilli described above—Dietzia included—can inhibit the MAP in culture extremely well, others moderately and others very poorly. Hence there is a variable ‘sensitivity’ of the numerous strains of MAP to the different inhibitory bacteria in the acid fast or mycolic acid possessing bacterial group. The Click strain did not work uniformly in all cattle as it also does not in all Crohn's patients. Hence one problem with the Click strain in relation to the treatment of Crohn's disease, is that it is capable of inhibiting many but not all MAP strains and it was chosen on the basis of inhibiting cattle MAP and not human MAP.

The original identification of acid fast and non-acid fast bacilli inhibiting cow MAP was detailed by W D Richards. ‘Environmental acidity may be the missing piece in the Johne's disease puzzle’, In ‘Johne's Disease’ 1989 Ed: A Milner and P Wood. CSIRO Publications, Melbourne. These interfering micro-organisms were considered contaminants during the culture of MAP from cow faeces. These cow faeces were collected from various veterinarian institutions, and he identified a number of acid fast and non-acid fast bacteria which he then co-cultured on slopes of media which could grow both MAP and the interfering strains of bacteria individually. MAP from cattle will grow readily on solid media and so this type of identification really applies particularly to Johnes' disease Mycobacteria. This methodology is not really suitable for identifying interfering bacteria with human MAP as the culture takes many months to grow.

### SUMMARY OF INVENTION

Provided are novel applications of bacteria which can profoundly interfere with bacteria generally belonging to the phylum of Actinobacteria to ameliorate or cure clinical infections caused by pathogens from this phylum such as those by Mycobacteriaceae and Mycobacterium such as M. tuberculosis. These organisms originate more specifically from the sub-order Corynebacterineae, family Dietziaceae, including genus Dietzia.

In alternative embodiments, provided are therapeutic combinations or consortiums of organisms comprising one or more species of the groups, orders or genus selected from the group consisting of: Actinobacteria, sub-order Corynebacterineae, genus Corynebacterium, Gordonia, Millisia, Skermania, Williamsia, Nocardiaceae, Rhodococcus, Smaradicoccus, Segniliparacae, Tsukamurellaceae, and any combination thereof. In alternative embodiments, the bacteria from the genus Corynebacterium is a Dietzia sp., optionally a specie as set forth in Table 1.

In alternative embodiments, provided are pharmaceutical compositions or formulations, or probiotic compositions, comprising the therapeutic combination as provided herein. In alternative embodiments, the pharmaceutical composition or formulation is formulated as an inhalant, or for oral administration, or formulated as a geltab or capsule, optionally an enterically coated capsule, or iceblock, or icecream, or optionally a multilayer capsule comprising the therapeutic combination in the inner layer.

In alternative embodiments, provided are uses of a therapeutic combination as provided herein, or a pharmaceutical composition or formulation, or a probiotic composition as provided herein, for the treatment, prevention, reversal of, or amelioration of: ulcerative colitis, Crohn's disease, collagenous colitis, microscopic colitis, lymphocytic colitis, pseudomembranous colitis, Clostridium difficile infection, diarrhoea or diarrhoea caused by Clostridium difficile infections, acute infective agents such as Salmonella, Shigella, Campylobacter, Aeromonas, Cholera and other acute gastrointestinal infections, infections which have an intracellular component, sarcoidosis, cardiac sarcoidosis, asthma, chronic H. pylori infection, irritable bowel syndrome, Type I and type II diabetes, psoriasis, multiple sclerosis (MS), obesity, infections of the lungs, cystic fibrosis, and/or Segniliparus (including S rugosus and rotundus) lung infections.

In alternative embodiments, provided are methods for the treatment, prevention, reversal of, or amelioration of: ulcerative colitis, Crohn's disease, collagenous colitis, microscopic colitis, lymphocytic colitis, pseudomembranous colitis, Clostridium difficile infection, diarrhoea or diarrhoea caused by Clostridium difficile infections, acute infective agents such as Salmonella, Shigella, Campylobacter, Aeromonas, Cholera and other acute gastrointestinal infections, infections which have an intracellular component, sarcoidosis, cardiac sarcoidosis, asthma, chronic H. pylori infection, irritable bowel syndrome, Type I and type II diabetes, psoriasis, multiple sclerosis (MS), obesity, infections of the lungs, cystic fibrosis, and/or Segnihparus (including S rugosus and rotundus) lung infections, comprising administering to an individual in need thereof a therapeutic combination as provided herein, or a pharmaceutical composition or formulation, or a probiotic composition as provided herein, wherein optionally the individual is a mammal, a human, or an animal, optionally a cattle or sheep.

In alternative embodiments, as provided herein, or the method as provided herein, further comprise administration of a fecal matter transplant (FMT) composition.

In alternative embodiments, provided herein is use of a combination or consortium of organisms comprising one or more species of the groups, orders or genus selected from the group consisting of: Actinobacteria, sub-order Corynebacterineae, genus Corynebacterium, Gordonia, Millisia, Skermania, Williamsia, Nocardiaceae, Rhodococcus, Smaradicoccus, Segnihparacae, Tsukamurellaceae, and any combination thereof, for the manufacture of a medicament for the treatment, prevention, reversal of, or amelioration of: ulcerative colitis, Crohn's disease, collagenous colitis, microscopic colitis, lymphocytic colitis, pseudomembranous colitis, Clostridium difficile infection, diarrhoea or diarrhoea caused by Clostridium difficile infections, acute infective agents such as Salmonella, Shigella, Campylobacter, Aeromonas, Cholera and other acute gastrointestinal infections, infections which have an intracellular component, sarcoidosis, cardiac sarcoidosis, asthma, chronic H. pylori infection, irritable bowel syndrome, Type I and type II diabetes, psoriasis, multiple sclerosis (MS), obesity, infections of the lungs, cystic fibrosis, and/or Segnihparus (including S rugosus and rotundus) lung infections.

In alternative embodiments, provided herein are methods for the treatment, prevention, reversal of, or amelioration of: ulcerative colitis, Crohn's disease, collagenous colitis, microscopic colitis, lymphocytic colitis, pseudomembranous colitis, Clostridium difficile infection, diarrhoea or diarrhoea caused by Clostridium difficile infections, acute infective agents such as Salmonella, Shigella, Campylobacter, Aeromonas, Cholera and other acute gastrointestinal infections, infections which have an intracellular component, sarcoidosis, cardiac sarcoidosis, asthma, chronic H. pylori infection, irritable bowel syndrome, Type I and type II diabetes, psoriasis, multiple sclerosis (MS), obesity, infections of the lungs, cystic fibrosis, and/or Segniliparus (including S rugosus and rotundus) lung infections, the method comprising:
(i) administering to an individual in need thereof a therapeutic combination, or pharmaceutical composition or formulation or probiotic composition (optionally single or combined strains where these may be alive and culturable, or killed) as described herein, for a period of time sufficient to obtain a desired therapeutic effect;
(ii) administering to said individual, after (i), a composition of antibiotics having anti-MAP activity (eg rifabutin, clofazimine, clarithromycin, metranidazole, ethambutol or mixtures thereof), optionally wherein said composition comprises anti-MAP antibiotics, for a period of time sufficient to obtain a desired therapeutic effect;
(iii) administering to said individual, after (ii), full spectrum fecal microbiota (FSM) implant, or cultures of single or mix of human gut microbiome bacteria or spores, sufficient to obtain a desired therapeutic effect.

In alternative embodiments of the method, the combination or composition described in (i) comprises at least one Dietzia sp., optionally a specie as set forth in Table 1.

In alternative embodiments of the method, each of steps (i), (ii), and (iii) is for a period of time, each independently selected, of between one and twelve weeks. The desired therapeutic effect could include reduction in symptoms such as any of diarrhoea, urgency, pain, bloating, rectal bleeding, fistula discharge, fevers and tenderness. A fall in the score of the Crohn's Disease Activity Index (CDAI) may be used to measure improvement, again describing the desired therapeutic effect, as can a fall in fecal calprotectin level.

In alternative embodiments of the method, in any one or more of (i), (ii), and (iii), the individual is administered the respective combination, composition, or implant on multiple occasions.

In alternative embodiments of the method, the method comprises multiple cycles of (i), (ii), and (iii), for example 2 cycles, or 3 cycles, or 4 cycles, or 5 cycles, or 6 cycles, or 7 cycles, or more.

In alternative embodiments of the method, administration of FSM is via colonoscopy, or via naso-gastric or naso-jejunal tube, or via enema.

In alternative embodiments of the method, the individual is a mammal, a human, or an animal, optionally a cattle or sheep. In alternative embodiments of the method the individual is a human.

In alternative embodiments of the method, the disease is Crohn's disease, colitis, indeterminate colitis, sarcoidosis, microscopic or collagenous colitis.

The details of one or more embodiments of the invention are set forth in the accompanying description below. Other features, objects, and advantages of the invention will be apparent from the description and the claims.

All publications, patents, patent applications cited herein are hereby expressly incorporated by reference for all purposes.

### BRIEF DESCRIPTION OF DRAWINGS

### DETAILED DESCRIPTION AND DESCRIPTION OF EMBODIMENTS

Provided are compositions and methods comprising use of bacteria of the Phylum Actinobacteria, sub-order Corynebacterineae genus Corynebacterium, within which reside Dietzia, and various other genera including Gordonia, Millisia, Skermania, Williamsia, Nocardiaceae, Rhodococcus, Smaradicoccus, Segniliparacae and Tsukamurellaceae.

This invention describes the surprising characteristic of the various non-pathogenic acid fast bacilli having a therapeutic power in inhibiting various Mycobacteria both in vitro and in vivo. In terms of the novel therapy as described herein, like bacteria from the same family will inhibit like bacterial members. In alternative embodiments, acid fast bacilli in the Actinobacteria phylum, e.g., Dietzia, inhibit in culture and in vivo the pathogenic mycobacteria that afflict man. In alternative embodiments strains of Dietzia, Rhodococcus, Nocardia, Gordonia, and other members of the genus of Corynebacterium are used to inhibit growth in culture and in vivo the various acid fast bacilli including mycobacteria, such as Mycobacterium avium subspecies paratuberculosis (MAP). These exemplary bacteria all contain mycolic acid in the cell walls which gives the bacterial walls a particular characteristic of being able to be stained with acid fast stain such as Ziehl-Neelsen stain and be able to live intracellularly.

In alternative embodiments, the genus Dietzia is used, and it stands out as a non-pathogenic genus with the largest number of potential organisms, and organisms from the genus Dietzia can be used singularly or in combination to inhibit the human-important infection with various mycobacteria; and alternative embodiments, exemplary organisms, are listed in Table 1:

TABLE 1

| | |
|---|---|
| Dietzia aerolata | Dietzia sp. ice-oil-79 |
| Dietzia alimentaria | Dietzia sp. II_Gauze_W_12-11 |
| Dietzia alimentaria 72 | Dietzia sp. IN108 |
| Dietzia aurantiaca | Dietzia sp. IN133 |
| Dietzia cercidiphylli | Dietzia sp. IR19 |
| Dietzia cf. maris V4.BE.23 | Dietzia sp. ISA13 |
| Dietzia cinnamea | Dietzia sp. ITRH56 |
| Dietzia cinnamea NBRC 102147 | Dietzia sp. J11R2A05 |
| Dietzia cinnamea | Dietzia sp. J4S14 |
| Dietzia dagingensis | Dietzia sp. J4S9 |
| Dietzia kunjamensis | Dietzia sp. J970 |
| Dietzia lutea | Dietzia sp. JC367 |
| Dietzia maris | Dietzia sp. JL-S7 |
| Dietzia natronolimnaea | Dietzia sp. JSM 077011 |
| Dietzia papillomatosis | Dietzia sp. JTS6048-306 |
| Dietzia papillomatosis NBRC 105045 | Dietzia sp. JTS6455-250 |
| Dietzia psychralcaliphila | Dietzia sp. JZDN52 |
| Dietzia schimae | Dietzia sp. K10S9 |
| Dietzia timorensis | Dietzia sp. K44 |
| Dietzia sp. ‘Mali 159’ | Dietzia sp. K6-17 |
| Dietzia sp. ‘Mali 88-02’ | Dietzia sp. KDB 1 |
| Dietzia sp. 02SU1 | Dietzia sp. KLBMP 1473 |
| Dietzia sp. 0705K4-1 | Dietzia sp. KNUC244 |
| Dietzia sp. 0711K6-1 | Dietzia sp. KNUC245 |
| Dietzia sp. 1-2 | Dietzia sp. KU03 |
| Dietzia sp. 1/4_C7/16_33 | Dietzia sp. KUA-5 |
| Dietzia sp. 100N22-1 | Dietzia sp. 117 |
| Dietzia sp. 100N22-3 | Dietzia sp. L21-PYE-C8 |
| Dietzia sp. 100N42-1 | Dietzia sp. LC021 |
| Dietzia sp. 101_(MB)_158mbsf | Dietzia sp. LC272 |
| Dietzia sp. 141_(MB)_32.2mbsf | Dietzia sp. LC367 |
| Dietzia sp. 147 | Dietzia sp. LC375 |
| Dietzia sp. 148 | Dietzia sp. LC376 |
| Dietzia sp. 14III/A01/021 | Dietzia sp. LC401 |
| Dietzia sp. 158Xa1 | Dietzia sp. LC431 |
| Dietzia sp. 168 | Dietzia sp. LH12 |
| Dietzia sp. 182_(MB)_89.1mbsf | Dietzia sp. LM0305 |
| Dietzia sp. 1R-10 | Dietzia sp. LOT4 |
| Dietzia sp. 2-2/G11 | Dietzia sp. M11-6-2 |
| Dietzia sp. 2216.35.9 | Dietzia sp. M1T8B24 |
| Dietzia sp. 241_(IO)_32.2mbsf | Dietzia sp. M2T8B1 |
| Dietzia sp. 291_(IO)_102mbsf | Dietzia sp. M2T8B4 |
| Dietzia sp. 3-149 | Dietzia sp. MBIC1537 |
| Dietzia sp. 3149 | Dietzia sp. MDT1-49-1 |
| Dietzia sp. 3372 | Dietzia sp. MG4 |
| Dietzia sp. 40 | Dietzia sp. MI-1.2 V3 |
| Dietzia sp. 41B_GOM-205m | Dietzia sp. MJ217 |
| Dietzia sp. 5IX/A01/142a | Dietzia sp. MJ624 |
| Dietzia sp. 61E40 | Dietzia sp. MJMG8.2 |
| Dietzia sp. 76 | Dietzia sp. MMRF600 |
| Dietzia sp. 7B_(MB)_50.2mbsf | Dietzia sp. MMRF603 |
| Dietzia sp. 8-57 | Dietzia sp. MMRF684 |
| Dietzia sp. a001-158 | Dietzia sp. MV04-01 |
| Dietzia sp. A1 | Dietzia sp. N11 |
| Dietzia sp. A103-104A | Dietzia sp. N1343 |
| Dietzia sp. A12 | Dietzia sp. N2 |
| Dietzia sp. A14101 | Dietzia sp. N21 |
| Dietzia sp. A1sdiesD4.2 | Dietzia sp. NB153 |
| Dietzia sp. A2 | Dietzia sp. NB252 |
| Dietzia sp. A3 | Dietzia sp. NITDS4 |
| Dietzia sp. a3(2010) | Dietzia sp. OB5 |
| Dietzia sp. A3(2014) | Dietzia sp. oral taxon 368 |
| Dietzia sp. Ac4 | Dietzia sp. oral taxon D12 |
| Dietzia sp. AD37 | Dietzia sp. P27-10 |
| Dietzia sp. AE45 | Dietzia sp. P27-19 |
| Dietzia sp. AS68 | Dietzia sp. P7.oi1.1 |
| Dietzia sp. AU645C | Dietzia sp. p9(2011) |
| Dietzia sp. B2/13 | Dietzia sp. Pazkelik11 |
| Dietzia sp. B3 | Dietzia sp. PCSB5 |
| Dietzia sp. BBDP42 | Dietzia sp. PD1 |
| Dietzia sp. BBDP47 | Dietzia sp. PDR22 |
| Dietzia sp. BBDP49 | Dietzia sp. PDR33 |
| Dietzia sp. BBDP51 | Dietzia sp. PDR4 |
| Dietzia sp. BJ-36 | Dietzia sp. PE-R2A-4 |
| Dietzia sp. BS1#2 | Dietzia sp. PETBA17 |
| Dietzia sp. BT20 | Dietzia sp. PJ-15 |
| Dietzia sp. BZ84 | Dietzia sp. PL005 |
| Dietzia sp. C-119 | Dietzia sp. PLB040 |
| Dietzia sp. C-22 | Dietzia sp. PLB051 |
| Dietzia sp. C7.oi1.2 | Dietzia sp. PLB073 |
| Dietzia sp. CA149 | Dietzia sp. PLB078 |
| Dietzia sp. Cai-32 | Dietzia sp. PLB113 |
| Dietzia sp. Cai-40 | Dietzia sp. PLB114 |
| Dietzia sp. CBMAI 705 | Dietzia sp. PLB123 |
| Dietzia sp. CCBAU 10911 | Dietzia sp. PLB132 |
| Dietzia sp. CH149b_4T | Dietzia sp. PmeaMuc17 |
| Dietzia sp. CH404b_13C | Dietzia sp. QAM_1_336 |
| Dietzia sp. Chol2 | Dietzia sp. qf11 |
| Dietzia sp. CIP104289 | Dietzia sp. R-23185 |
| Dietzia sp. CIP104293 | Dietzia sp. R144 |
| Dietzia sp. CKS_01 | Dietzia sp. R18 |
| Dietzia sp. CN-3 | Dietzia sp. R19 |
| Dietzia sp. CNJ898 PL04 | Dietzia sp. R23 |
| Dietzia sp. CO99 | Dietzia sp. R30 |
| Dietzia sp. COL-66 | Dietzia sp. R32 |
| Dietzia sp. COS1 | Dietzia sp. Rc12a |
| Dietzia sp. CQ4 | Dietzia sp. RKEM 832 |
| Dietzia sp. CR1-3 | Dietzia sp. RMS10 |
| Dietzia sp. CUA-696 | Dietzia sp. S-JS-1 |
| Dietzia sp. CW-19 | Dietzia sp. S-XJ-2 |
| Dietzia sp. CW-21 | Dietzia sp. S1-38 |
| Dietzia sp. CY-b19 | Dietzia sp. S3 |
| Dietzia sp. CY-b30 | Dietzia sp. SB2 |
| Dietzia sp. D3 | Dietzia sp. SBP310 |
| Dietzia sp. d30 | Dietzia sp. SBT353 |
| Dietzia sp. D5 | Dietzia sp. SBT354 |
| Dietzia sp. DQ12-45-1b | Dietzia sp. SBT355 |
| Dietzia sp. DTS-26 | Dietzia sp. SCULCB HNA-3 |
| Dietzia sp. E1 | Dietzia sp. SG-3 |
| Dietzia sp. E241 | Dietzia sp. SGD-1011 |
| Dietzia sp. E34D | Dietzia sp. SK79 |
| Dietzia sp. E9_2 | Dietzia sp. SLG510A3-17 |
| Dietzia sp. EBKC103 | Dietzia sp. SNRW2-1 |
| Dietzia sp. EBKC115 | Dietzia sp. SU24 |
| Dietzia sp. EBKC116 | Dietzia sp. SUB2 |
| Dietzia sp. EBKC15 | Dietzia sp. Taihu-001 |
| Dietzia sp. EBKC36 | Dietzia sp. Tc3-16 |
| Dietzia sp. EBKC47 | Dietzia sp. TmT3-14-1 |
| Dietzia sp. EBKC80 | Dietzia sp. UCD-THP |
| Dietzia sp. EBKC9 | Dietzia sp. UmPM_1_364 |
| Dietzia sp. EBKC92 | Dietzia sp. URC-0-5 |
| Dietzia sp. EBKC96 | Dietzia sp. UT 1-05 |
| Dietzia sp. EF2B-B525 | Dietzia sp. UW-23 |
| Dietzia sp. EGI 80187 | Dietzia sp. VF38-3 |
| Dietzia sp. EGI80084 | Dietzia sp. VG23-2 |
| Dietzia sp. ES-QY-1 | Dietzia sp. VH37-3 |
| Dietzia sp. ES18 | Dietzia sp. VI37-3 |
| Dietzia sp. F09TDL | Dietzia sp. VI38-3 |
| Dietzia sp. f10(2011) | Dietzia sp. VN1-3 |
| Dietzia sp. F148 | Dietzia sp. VN3-3 |
| Dietzia sp. F152M | Dietzia sp. VN4-3 |
| Dietzia sp. f18(2011) | Dietzia sp. VP6-3 |
| Dietzia sp. f5(2011) | Dietzia sp. VR5-3 |
| Dietzia sp. f8(2011) | Dietzia sp. VS3-2 |
| Dietzia sp. FB10 | Dietzia sp. W02TDL |
| Dietzia sp. FI 1026 | Dietzia sp. W5004 |
| Dietzia sp. FP004 | Dietzia sp. W5026 |
| Dietzia sp. FS36 | Dietzia sp. WLSH-60 |
| Dietzia sp. FXJ8.094 | Dietzia sp. WR-3 |
| Dietzia sp. FXJ8.156 | Dietzia sp. X-b1 |
| Dietzia sp. FXJ8.228 | Dietzia sp. X-c3 |
| Dietzia sp. g3 | Dietzia sp. XSW067 |
| Dietzia sp. GN107 | Dietzia sp. Y3 |
| Dietzia sp. F09TDL | Dietzia sp. Y32 |
| Dietzia sp. f10(2011) | Dietzia sp. YB228 |
| Dietzia sp. GN24 | Dietzia sp. YIM 100291 |
| Dietzia sp. GN50 | Dietzia sp. YIM 64718 |
| Dietzia sp. GN53 | Dietzia sp. YIM 68234 |
| Dietzia sp. GN67 | Dietzia sp. YIM 75753 |
| Dietzia sp. GN68 | Dietzia sp. YIM 76027 |
| Dietzia sp. GN722 | Dietzia sp. YIM M10497 |
| Dietzia sp. GN78 | Dietzia sp. YL-1 |
| Dietzia sp. GPM2604 | Dietzia sp. YMF_0365 |
| Dietzia sp. H0 | Dietzia sp. YMF_1348 |
| Dietzia sp. H05TDL | Dietzia sp. Z140 |
| Dietzia sp. H0B | Dietzia sp. Z306 |
| Dietzia sp. H202 | Dietzia sp. ZAL-04 |
| Dietzia sp. H2f | Dietzia sp. zf-IIRht6 |
| Dietzia sp. HBUD30513 | Dietzia sp. ZJY-402 |
| Dietzia sp. HMSC21D01 | Dietzia sp. ZJY-430 |
| Dietzia sp. HRJ2 | Dietzia sp. ZQ-4 |
| Dietzia sp. HRJ3 | environmental samples |
| Dietzia sp. HZBC62 | Dietzia sp. enrichment culture |
| Dietzia sp. I-B03 | Dietzia sp. enrichment culture |
| Dietzia sp. I_GA_W_11_7 | clone MWF-14-6-10-27F |
| Dietzia sp. IBT6499-C01 | uncultured Dietzia sp. |
| Dietzia sp. ice-oil-101 | unclassified Dietziaceae |
| Dietzia sp. ice-oil-124 | Dietziaceae bacterium SM30 |
| Dietzia sp. ice-oil-71 | Dietziaceae bacterium SM37 |

Because the Dietzia are largely innocuous, in alternative embodiment they are also used in children, e.g., children whose cystic fibrosis disease is often super-infected by non-tuberculosis mycobacteria (NTM) especially Mycobacterium abscessus complex (MABSC), and Mycobacterium avium complex (MAC). These can infect insidiously and cause serious morbidity and mortality in children with cystic fibrosis.

In alternative embodiments, provided are compositions comprising probiotic acid fast bacilli/mycolic acid-containing bacteria which can be administered to patients to inhibit the intracellular pathogens responsible for the diseases as described herein. In alternative embodiments, in clinical applications, e.g., where numerous patients are treated, each carrying different Mycobacterium avium subspecies paratuberculosis (MAP) strains, we have found that numerous Dietzia bacteria each with different inhibitory capability need to be combined to result in a more powerful inhibition of almost all known strains of MAP and other pathogens. Hence, provided are combinations of MAP-inhibiting Dietzia bacteria capable of treating more effectively different MAP strains so that few Crohn's MAP strains will not be covered by their inhibition of MAP growth. This is analogous to using combined antibiotics to achieve cure of stubborn bacteria.

Provided are methods for the identification of interfering or therapeutic bacterial strains, which by practicing methods are provided herein can be selected in a rational manner and combined in groups of inhibitory bacteria, e.g., a group of Dietzia or a group of Rhodococcus or various mixtures—so that the group will be able to inhibit numerous strains, e.g., pathogenic bacterial strains, e.g., of cow, sheep or human Mycobacterium avium paratuberculosis and their subspecies. The reason for combining a number of the organisms is to cover the various ‘sensitivities’ of MAP strains that are found in different patients with Crohn's or sarcoidosis. Therapeutic combinations for treating other human mycobacterial conditions are also identified and provided, including e.g., therapeutic combinations for treating resistant Mycobacterium tuberculosis, leprosy, atypical lung infections with Mycobacterium avium avium and MAC, skin and abscess infections with the various atypical mycobacteria. Provided are methods for the specific identification of groups of interfering acid fast mycolic acid-containing bacteria which are individually selected and combined in a composition (e.g., a pharmaceutical combination, or a probiotic as provided herein) that would give the broadest cover to inhibit as many as possible of the various pathogenic strains detected clinically. Hence, rather than, for example, inhibiting MAP in only one third of Crohn's patients, by practicing methods as provided herein therapeutic combinations for treating MAP in most, if not all of Crohn's patients are identified. Methods provided herein address the need for providing therapeutic combinations for treating all or most of even several MAP strains within the one Crohn's patient; and in alternative embodiments, methods provided herein select the appropriate therapeutic combination of Dietzia, Rhodococcus or Nocardia strains (to mention a few).

The invention will be further described with reference to the following examples; however, it is to be understood that the invention is not limited to such examples.

### EXAMPLE 1

### Selection of Map-Interfering Bacteria from Cattle

The following example describes exemplary methods for the selection of therapeutic combinations of interfering bacteria to treat Mycobacterium avium subspecies paratuberculosis (MAP) in cattle.

MAP can be cultured from numerous sources to cover various cattle, preferably across a number of farms and a number of countries. The blood from such cows can be divided into several different tubes and various concentrations of (for example) Dietzia from 10² through to 10¹⁵ would be added to several tubes but only the saline carrier would be added to the control tube. After incubation for 8 or 20 days, MAP proliferation within stained macrophages will be examined under the microscope to see whether the particular Dietzia selected from that particular cow is inhibiting the MAP. The microscope screening test using macrophage proliferation of MAP saves much time otherwise required for MAP grown in culture.

The next stage would be to culture on slopes appropriate for MAP culture and co-culture with a Dietzia strain. This then can be set up to test multiple strains e.g. ten different strains of Dietzia—and find which strains are the most powerful MAP inhibitors. This can then be repeated with numerous strains of MAP to make sure that all the clinical strains of MAP can be inhibited by that Dietzia organism or by other candidate organisms e.g., Rhodococcus. From current experience, it is expected that at least 6-10 Dietzia strains will be required to cover the great majority of culturable MAP strains from cattle.

This would then be used as a probiotic fed orally to cattle to inhibit the infection residing in various cows. Monitoring would be of antibody levels to MAP, body mass, and output of MAP in stools in the cattle. The other method would be to follow macrophage multiplication of MAP in the blood by serial examination allowing the macrophages to cultivate and be examined and stained under the microscope with modified Ziehl-Neelsen stain.

### EXAMPLE 2

### Selection of Human Anti-Map Interfering Strains

The following example describes exemplary methods for the selection of therapeutic combinations of interfering bacteria to treat Mycobacterium avium subspecies paratuberculosis (MAP) in humans.

Patients with Crohn's disease will have their blood collected and macrophage strains of MAP are cultured over 10 -20 days in the presence and in the absence of Dietzia or other candidate inhibitory strains, for example, strains selected from the process as described in Example 1. Numerous Dietzia strains are tested for each patient and then the most effective Dietzia inhibitors are combined in a group of 6-10 Dietzia strains to be used as an oral therapeutic agent. The larger the number of human strains that are co-incubated with the Dietzia, the greater the cover of probiotic inhibition will be in the treatment of Crohn's disease. Rising concentrations of Dietzia within the blood incubation will also help determine the titre at which the Dietzia will start inhibiting the infecting MAP. Here one can use growth of MAP in macrophages to identify the most potent inhibitory species.

In alternative embodiments, just as one or more Dietzia or Rhodococcus are used as the inhibitory strain or strains, other mycolic acid-containing non-pathogenic bacteria can be substituted for Dietzia or used in addition to Dietzia to create a therapeutic combination. These include various strains of the Dietzia clade, and other genera such as Gordonia (e.g., as listed in Table 4, below), Nocardia (e.g., as listed in Table 5, below), Millisia (M brevis-J81T and J82), Nocardia, Smaragdicoccus, (including Smaragdicoccus niigatensis), Streptomyces (over 576 species), Skermania (including S piniformis), Turicella (including Totitidis), Tsukamurella (e.g., as listed in Table 6, below), Segniliparus (including S rugosus and rotundus), Corynebacterium (e.g., as listed in Table 2, below), Rhodococcus (e.g., as listed in Table 3, below) and Williamsia (e.g., as listed in Table 7, below). All can be used as groups of multiple strains of the same genus, or mixtures of various genera contingent on sensitivity results. Furthermore, in the individual patient with difficult-to-inhibit MAP or those with more than one strain, custom-built combinations can be assembled for more effective treatment.

TABLE 2
(Corynebacterium)

| | |
|---|---|
| C. accolens | C. matruchotii |
| C. afermentans | C. minutissimum |
| C. ammoniagenes | C. parvum (Propionibacterium acnes) |
| C. amycolatum | C. paurometabolum |
| C. argentoratense | C. propinquum |
| C. aquaticum | C. pseudodiphtheriticum (C. hofmannii) |
| C. auris | C. pseudotuberculosis |
| C. bovis | (C. ovis) |
| C. equi (now Rhodococcus equi) | C. pyogenes-Trueperella pyogenes |
| C. flavescens | C. urealyticum (group D2) |
| C. glucuronolyticum | C. renale |
| C. glutamicum | C. spec |
| C. granulosum | C. striatum |
| C. haemolyticum | C. tenuis |
| C. halofytica | C. ulcerans |
| C. kroppenstedtii | C. urealyticum |
| C. jeikeium (group JK) | C. xerosis |
| C. macginleyi | |

TABLE 3
(Rhodococcus)

| | |
|---|---|
| Rhodococcus aurantiacus | Rhodococcus opacus |
| Rhodococcus baikonurensis | Rhodococcus percolatus |
| Rhodococcus boritolerans | Rhodococcus phenolicus |
| Rhodococcus equi | Rhodococcus polyvorum |
| Rhodococcus coprophilus | Rhodococcus pyridinivorans |
| Rhodococcus corynebacterioides | Rhodococcus rhodochrous |
| (synonym: | Rhodococcus rhodnii |
| Nocardia corynebacterioides) | (synonym: |
| Rhodococcus erythropolis | Nocardia rhodnii) |
| Rhodococcus fascians (synonym: | Rhodococcus ruber (synonym: |
| Rhodococcus luteus) | Streptothrix rubra) |
| Rhodococcus globerulus | Rhodococcus jostii RHA1 |
| Rhodococcus gordoniae | Rhodococcus triatomae |
| Rhodococcus jostii | Rhodococcus tukisamuensis |
| Rhodococcus koreensis | Rhodococcus wratislaviensis (synonym: |
| Rhodococcus kroppenstedtii | Tsukamurella wratislaviensis) |
| Rhodococcus maanshanensis | Rhodococcus yunnanensis |
| Rhodococcus marinonascens | Rhodococcus zopfii |

TABLE 4
(Gordonia)

| | |
|---|---|
| G. aichiensis | G. paraffinivorans |
| G. alkanivorans | G. polyisoprenivorans |
| G. amarae | G. rhizosphera |
| G. amicalis | G. rubripertincta |
| G. bronchialis | G. sihwensis |
| G. desulfuricans | G. sinesedis |
| G. hirsuta | G. spumae |
| G. hydrophobica | G. sputi |
| G. jacobaea | G. terrae |
| G. namibiensis | G. westfalica |
| G. nitida | |

TABLE 5
(Nocardia)

| | |
|---|---|
| N. aerocoloninges | N. farcinica |
| N. africana | N. nigiitansis |
| N. argentinensis | N. nova |
| N. asteroides | N. opaca |
| N. blackwellii | N. otitidis-cavarium (previously N. caviae) |
| N. brasiliensis | N. paucivorans |
| N. brevicatena | N. pseudobrasiliensis |
| N. carnea | N. rubra |
| N. cerradoensis | N. seriolae |
| N. corallina | N. transvelencesis |
| N. cyriacigeorgica | N. uniformis |
| N. dassonvillei | N. vaccinii |
| N. elegans | N. veterana |

TABLE 6
(Tsukamurella)

| | |
|---|---|
| T. inchonensis | T. spumae |
| T. paurometabola | T. strandjordii |
| T. pseudospumae | T. tyrosinosolvens |
| T. pulmonis | T. wratislaviensis |

TABLE 7
(Williamsia)

| | |
|---|---|
| W muralis | W maris |
| W daligens | W phyllosphaerae |
| W faeni | W serinedens |
| W limnetica | W sterculiae |
| W marianensis | |

Once the patient's mycobacteria circulating in macrophages have been inhibited, it is confirmed that the particular added mycolic acid-containing the inhibitory bacteria, or mix of bacteria,—the exemplary therapeutic combination—are working against that humanised MAP. The best inhibitory bacteria are then collected after studying a number of patients with Crohn's disease, so that a good cross-section of the best inhibitory non-pathogenic acid-fast bacilli can be used.

### EXAMPLE 3

### Selection of Map-Interfering Bacteria from Sheep

The following example describes exemplary methods for the selection of therapeutic combinations of interfering bacteria to treat Mycobacterium avium species paratuberculosis (MAP) in sheep (Johne's disease in sheep).

In similar fashion as described for both humans and cattle, the same exemplary process can be repeated in sheep. However, in both cattle and sheep, the MAP can be actually cultured in the laboratory and hence the interference of the suppressing bacterium can be tested against sheep and cattle MAP somewhat more easily that it can with human MAP, which will not grow that readily in a laboratory using solid culture media or even liquid culture medium.

In alternative embodiments, methods of culture of the sheep and cattle MAP—and same applies to the growth of Mycobacterium tuberculosis—on HEYM slants and also modified Middlebrook 7H10 Agar medium can be used to create slants for MAP. These media in screw-top test tubes will therefore contain Mycobacterium paratuberculosis. Two weeks or more after culturing the MAP on the slants, the inhibitory bacteria can be inoculated as spots on the slants. They can be inoculated in various dilutions to study the power of inhibition of even low dilutions. Once the slants are spotted they are incubated at 37 degrees C. in an aerobic jar with carbon dioxide (a jar charged with carbon dioxide). Prolonged incubation may be required for the MAP from sheep and cattle that may need 8 to 12 weeks of incubation, but if the MAP does not appear even after prolonged incubation it indicates that the spotted inhibitors achieved total inhibition of the cultured MAP. If there is no inhibition then MAP will be seen growing.

For both cattle and sheep, both the blood and testing inhibition and slant co-culture can be used to select the best organism to inhibit MAP growth.

### EXAMPLE 4

### Selection of Human Anti-Map Interfering Strains Custom Built for a Patient

The following example describes identification and selection of therapeutic combinations of human anti-MAP bacteria.

In some situations there will be Crohn's patients who have an unusual genetic structure of their MAP bacterium resistant to the commercial Anti-MAP probiotics combination therapeutic. In this patient, stored interfering bacteria e.g., Rhodococcus Dietzia or Nocardia, may be co-cultured with the patient's blood to determine which of these will inhibit growth of the MAP in the macrophages. This patient may not respond to a standardised mix of the anti-MAP probiotics that might be commercially available, but rather may have to go through the process of selecting a unique therapeutic combination of Dietzia and other MAP inhibitors, including strains as described in Example 2, e.g., strains in storage by a laboratory that builds individualised or customised anti-MAP Probiotics, e.g., from a group of mycolic acid containing bacteria.

### EXAMPLE 5

### Combination Therapy of the Mycolic Acid-Containing Acid Fast Bacteria Combined with Full Spectum Microbiota Implantation or Administration

The following example describes an exemplary combination therapy and a therapeutic combination comprising mycolic acid-containing acid fast bacteria (listed above in Example 2) combined with full spectrum microbiotia, e.g., full spectrum fecal microbiota, implantation or administration, e.g., by oral administration, e.g., as a liquid, in capsules and the like.

Crohn's disease ulcerative colitis and other inflammatory conditions in the bowel may require a combination of: a. replacement flora to restore missing components such as Bacteroides or Firmicutes; and the patient may also require the presence of b. MAP-inhibitory consortium or therapeutic combination of organisms, e.g., MAP-inhibitory consortium or therapeutic combinations as provided herein.

In this situation, purified and concentrated donor flora as described in other patent applications (Sadowsky et al 2015; US 2015/0374761; Borody T J 2015; US 2015/0297642) is lyophilised and manufactured into capsules and can be used in conjunction with lyophilised capsules or liquid drinks of the exemplary therapeutic combination of anti-human MAP-inhibitory bacteria as provided herein.

This exemplary therapeutic combination improves on use of full spectrum microbiota alone, where full spectrum microbiota administration can in itself inhibit Crohn's disease and is some situations end up with a cure (see e.g., Borody et al. Fecal Microbiota Transplantation. Gastroenterol Clin N Am 2012;781-803). Use of Dietzia, using multiple strain, and even a single strain, can very quickly put patients with Crohn's disease into remission.

The inventor's clinical experience with treatment of 6 Crohn's patients for more than 10 weeks with a single Dietzia strain has shown 6/6 patients going to fairly rapid remission within 2-3 weeks of treatment, with the Crohn's Disease Activity Index or CDAI, falling from average of 300 points to less than 150. Patients' abdominal pain, cramping, diarrhoea and urgency progressively abate and even their joint pain improved, and they gained weight.

Exemplary therapeutic combinations as provided herein can be powerful and first-line therapies for Crohn's as soon as it is diagnosed in clinical practice. For example, patients may be able to avoid steroid use, immunosuppressant's anti-inflammatory agents, anti-TNF alpha products and other more dangerous agents. Patients can be placed into immediate therapy using this exemplary therapeutic combination, which is a powerful Crohn's treatment.

In one embodiment, the exemplary therapeutic combination comprises mycolic acid containing acid fast bacteria as a group together with full spectrum Microbiota, and in one exemplary method administration is on a daily basis taken either once, twice or many times during the day to ensure passage of this army of various inhibitory bacteria through the gastrointestinal tract. This exemplary therapeutic combination as an inhibitory therapy also may be necessary for the rare case of patients who do not respond to any medications. So in summary, this is a combination of purified human donor bacteria together with eg active strains of Dietzia encapsulated as a prolonged oral therapy.

### EXAMPLE 6

### Combination of Synthetic (cultured ) or Cut-Down Versions of Full Spectrum Microbiota together with Anti-Human Map-Inhibitory Bacteria

In alternative embodiments, in therapeutic combinations provided herein, the full spectrum microbiota (FSM) may be substituted by cultured bacteria comprising the various relevant organisms found in the human gut microbiome. These could include Firmicutes, Bacteroidetes, Actinobacteria, Acidobacteria, Chlamydiae Cyanobacteria, Deferribacteres, Deionococcus-Thermus, Dictyoglomi, Fibrobacteres, Fusobacteria, Gemmatimonadetes, Nitrospirae, Planctomyces, Proteobacteria, Spirochaetes, Thermodesulfobacteria, Thermomicrobia,Thermotogae, and Verrucomicrobia. Furthermore spores alone can be used instead of vegetative forms of Firmicutes or Bacillus to constitute the MAP-inhibiting bacteria. As with the treatment of C. difficile, one can use only a small number of strongly inhibiting bacteria such as Clostridia in their vegetative forms or as spores in combination with the mycolic acid MAP-inhibitory bacteria as delineated above. This permits a smaller volume of implanted bacteria as spores, but still accompanied by the powerful MAP-inhibitory bacteria such as Dietzia, Rhodococcus or other Actinobactreia.

### EXAMPLE 7

### Order of Treatment using Fecal Matter Transplants (FMT) as Full Spectrum Microbiota (FSM) or as Small Consortium of Extracted or Cultured Probiotics Plus Map-Inhibiting Bacteria

In a further refinement the physical order of administration of the therapies can play a significant role in clinical response. The described therapies, including FMT, small versions of FMT, standard anti-Crohn's therapies, Anti-MAP bacterial treatments, anti-MAP vaccine and Anti-MAP bacteria—need to be lined up in such an order that they achieve the best result—preferably a cure of Crohn's disease or of Colitis. In this example the order of administration can be crucial especially in the very ill Crohn's patient who is anemic, has fistulae, has had surgery, and/or may have a stoma.

Generally this type of patient requires rapid induction of remission (fall in CDAI or Harvey-Bradshaw Index score). This can be achieved by administration of steroids, short-term anti-TNF treatment e.g. 3 infusions, and use of anti-MAP antibiotics, such as rifabutin, clarithromycin, clofazimine, metronidazole, ethambutol, ciprofloxacin, pyrazinamide or others already in the public domain. As the patient improves and stool frequency falls, pain improves and haemoglobin rises, then the combination of FSM+Anti-MAP bacteria in a single capsule can be administered. Soon after, one can commence use of Anti-MAP vaccine to stimulate the body's immunity against MAP. Nevertheless the duration of the oral capsules of FMT+Anti-MAP bacterial treatment will likely continue for many months or even years, monitored by colonoscopic healing progress. In milder disease and in colitis immediate use of the FMT+Anti-MAP bacteria in capsules can be commenced upon diagnosis. Monitoring the circulating macrophages for diminishing growth of stainable MAP together with recording clinical improvement will give an idea of the healing taking place at the mucosal level.

In addition to providing a greater and more powerful ability for treating Crohn's disease and ulcerative colitis, exemplary therapeutic combinations provided herein are also useful for other indications and various formats of treatment, for example:

1. The patient with frequent diarrhoea, bloating and urgency and mucus: 1^{st}: can initially have the symptoms rapidly controlled with the treatments comprising Prednisone or other steroids such as Budesonide, and other antibiotics to quickly bring the patient under clinical control. So agents such as Rifaximin, Aztreonam, Rifabutin, Rifampin, Vancomycin, Gentamicin, Streptomycin, or other non-absorbable agents can be combined or used simply to quickly reduce the symptoms in the patient. 2^{nd} The next treatment will be cessation of antibiotics and serial ingestion of full spectrum Microbiota lyophilised in capsules. This would be used to inhibit or eradicate such as agents as Clostridium difficile, MRSA, VRE, and resistant Klebsiella. 3^{rd} Finally the patient will then be treated for prolonged periods of time for months or years with the added composition of the mycolic acid-containing anti-MAP inhibitory organisms, as described above.

2. Patients with fairly mild disease would avoid the need for antibiotics and steroids but would be commenced with use of simultaneous composition of the anti-MAP inhibitor bacteria and full spectrum Microbiota either in separate capsules or in a same capsule. The medication can be delivered daily, twice daily, three times daily or as required and could also be used by other routes of administration such as nasojejunal tube, enema or through a stoma in unusual situations.

3. The method of usage of this medication in point 2 can then be supplemented by immunisation against MAP to create one's own immune resistance and not have to keep taking expensive capsules of the anti-MAP and full spectrum Microbiota but rather stay immunised and be re-immunised perhaps monthly, 6 monthly or yearly.

### EXAMPLE 8

### Use of Stool Donor as Reactor for Creating Anti-MAP Bacterial Inhibitors in Combination with Full Spectrum Microbiota (FSM) to Serve as a Therapeutic

Full spectrum microbiota is obtained by collecting donor stool filtering out the non-bacterial components and lyophilisating the pure suspension of the multiple phyla of bacteria in human flora. The full spectrum microbiota is collected from donor stools because the human body is a factory or incubator for producing full spectrum microbiota. This fact can be utilised to produce a super FSM by feeding the donors appropriate harmless microbacteria inhibitory mycolic acid bacteria such as Dietzia. As the patient eats the Dietzia, its presence and concentration can be measured, e.g., its presence can be found in a stool sample. The donor is therefore producing a mix of full spectrum microbiota together with a MAP-inhibitory agent or agents. Increasing the number of the anti-MAP inhibitory bacteria can produce a targeted FSM donated from an anti-Crohn's donor stool. For example, a donor can be fed 6 different bacterial strains of such genera as Rhodococcus, Dietzia or whatever one has chosen in vitro to work in Crohn's. The inhibitory mycolic acid-containing bacteria are passed in the stool and the entire stool is homogenised and filtered and is ready for encapsulation to treat the various conditions listed, particularly Crohn's disease.

The healthy donor who donates stool for the production of FSM or its cut-down products is known from our experiments to have detectable Dietzia strains in stool upon feeding Dietzia orally. This phenomenon continues for up to 4-6 weeks after cessation of feeding. In this way the donor is a ‘reactor’ in whom the combination of a FSM and e.g. Dietzia co-exist in the donated stool and can be processed to a lyophilised capsule—which can be used as a therapy for C. difficile, MRSA, VRE as well as Crohn's disease and Ulcerative colitis. This is a manufacturing ‘short cut’ resulting in an ideal combination therapy made safer by being made within a donor gastrointestinal tract.

Such a product can be further optimised within the donor by incubating the stool components by use of cooler environment, altering the diet, and addition of trehalose in which Dietzia flourishes, and later use of aerobic atmosphere to enhance Dietzia numbers when the donated stool is placed in an incubator with a cooler temperature and added oxygen.

Meanwhile the other portion of the donated stool can be incubated in an anaerobic atmosphere to enhance the anaerobic components then later combine both of these and so create a product for lyophilisation with higher Dietzia composition. Further addition of spores, extracted recurrently from the donor's separate donated stool, using the alcohol extraction procedure, can be used to markedly supplement the product ending up with high Dietzia and high spore composition, as well as high Bacteroides and Firmicutes populations. One or more organisms listed in Tables 1-8 can be fed to the donor so producing a donor super FSM. Feeding the donor friendly compounds used as culture components for these probiotics can further enhance the numbers of the Anti-MAP probiotics in the donated stool.

### EXAMPLE 9

### Cycling Combination Treatment

Having learned that Dietzia and other mycolic acid-containing bacteria initially accelerate MAP growth intracellularly, it makes sense to follow this pre-treatment with Anti-MAP antibiotics. Since antibiotics directed at MAP also affect the GI microbiome, it would then be best to restore the gut flora using FSM fecal implant.

Hence we set out to treat with Dietzia initially, for 3 weeks then switched to Anti-MAP for 4 weeks, then completed with 2 weeks of FSM via colonoscopy then enema.

This cycling was continued for 3 cycles, and at the last colonoscopy the progressive healing of the colonic Crohn's disease was all but complete with only several pseudopolyps remaining. Both living and dead Dietzia or other mycolic acid containing probiotics in this class, can be used with good effect.

### Conditions Treated

In alternative embodiments, a number of conditions are treatable, prevented or ameliorated using exemplary therapeutic combinations provided herein, for example, these include ulcerative colitis, Crohn's disease, collagenous colitis, microscopic colitis, lymphocytic colitis, pseudomembranous colitis, Clostridium difficile and diarrhoea, or Clostridium difficile infections, acute infective agents such as Salmonella, Shigella, Campylobacter, Aeromonas, Cholera and other acute gastrointestinal infections.

In alternative embodiments, exemplary therapeutic combinations provided herein are useful for treating and ameliorating many different infections, e.g., infections which have an intracellular component. For example, exemplary therapeutic combinations can be used to treat sarcoidosis, which is known to be associated with the presence of MAP, and in on embodiment, cardiac sarcoidosis which is difficult to access otherwise.

In alternative embodiments, exemplary therapeutic combinations provided herein are useful for treating and ameliorating asthma and chronic H. pylori infection.

In alternative embodiments, exemplary therapeutic combinations provided herein are useful for treating and ameliorating irritable bowel syndrome, Type I and type II diabetes, psoriasis, MS and obesity as there is evidence that MAP is associated with these conditions.

In alternative embodiments, exemplary therapeutic combinations provided herein are useful for treating and ameliorating tuberculosis, including various tuberculous-causative agents. For example, Mycobacterium tuberculosis infection, particularly the resistant strains, also are amenable to treatment by exemplary Dietzia agents and similar mycolic acid containing bacteria combinations as provided herein, or identified by methods provided herein.

Many of the atypical Mycobacteria remain chronic in patients whether they immunocompromised or not, and antibiotics that are generally used first are found not to progress treatment; and further reversal of the condition will need still to be stopped. In alternative embodiments, exemplary therapeutic combinations provided herein are useful for treating and ameliorating atypical Mycobacterial infections. For example, in alternative embodiments, patients are given exemplary therapeutic combinations comprising mycolic acid containing inhibitory bacteria as listed above e.g. Dietzia Rhodococcus, Nocardia and others. An exemplary composition comprising such anti-MAP bacteria may contain 10² to 10¹⁴ bacteria; in some situations would probably be the best to start with. Individualised or custom built treatment to a cultured atypical Mycobacterium , e.g. Mycobacterium avium species, by practicing methods as provided herein, could then be designed and produced if the patient does not respond adequately to the standard mix.

Other severe infections of the lungs, e.g., in patients with cystic fibrosis, that may carry Segniliparus (including S rugosus and rotundus), can be treated using exemplary inhaled anti-MAP Rhodococcus or Dietzia or other combinations as provided herein, for example, to inhibit their growth in the bronchi where antibiotics have failed.

A number of embodiments of the invention have been described. Nevertheless, it will be understood that various modifications may be made without departing from the spirit and scope of the invention. Accordingly, other embodiments are within the scope of the following claims.

1. A therapeutic combination of organisms comprising one or more species of the groups, orders or genus selected from the group consisting of: Actinobacteria, sub-order Corynebacterineae, genus Corynebacterium, Gordonia, Millisia, Skermania, Williamsia, Nocardiaceae, Rhodococcus, Smaradicoccus, Segnihparacae, Tsukamurellaceae, and any combination thereof.

2. The therapeutic combination of claim 1, wherein the bacteria from the genus Corynebacterium is a Dietzia sp., optionally a specie as set forth in Table 1.

3. A pharmaceutical composition or formulation, or a probiotic composition, comprising the therapeutic combination of claim 1 or claim 2.

4. The pharmaceutical composition or formulation of claim 3, formulated as an inhalant, or for oral administration, or formulated as a geltab or capsule, optionally an enterically coated capsule, iceblock, icecream, or optionally a multilayer capsule comprising the therapeutic combination in the inner layer.

5. Use of a therapeutic combination of claim 1 or claim 2, or a pharmaceutical composition or formulation, or a probiotic composition of claim 3 or claim 4, for the treatment, prevention, reversal of, or amelioration of: ulcerative colitis, Crohn's disease, collagenous colitis, microscopic colitis, lymphocytic colitis, pseudomembranous colitis, Clostridium difficile infection, diarrhoea or diarrhoea caused by Clostridium difficile infections, acute infective agents such as Salmonella, Shigella, Campylobacter, Aeromonas, Cholera and other acute gastrointestinal infections, infections which have an intracellular component, sarcoidosis, cardiac sarcoidosis, asthma, chronic H. pylori infection, irritable bowel syndrome, Type I and type II diabetes, psoriasis, multiple sclerosis (MS), obesity, infections of the lungs, cystic fibrosis, and/or Segnihparus (including S rugosus and rotundus) lung infections.

6. A method for the treatment, prevention, reversal of, or amelioration of: ulcerative colitis, Crohn's disease, collagenous colitis, microscopic colitis, lymphocytic colitis, pseudomembranous colitis, Clostridium difficile infection, diarrhoea or diarrhoea caused by Clostridium difficile infections, acute infective agents such as Salmonella, Shigella, Campylobacter, Aeromonas, Cholera and other acute gastrointestinal infections, infections which have an intracellular component, sarcoidosis, cardiac sarcoidosis, asthma, chronic H. pylori infection, irritable bowel syndrome, Type I and type II diabetes, psoriasis, multiple sclerosis (MS), obesity, infections of the lungs, cystic fibrosis, and/or Segnihparus (including S rugosus and rotundus) lung infections, comprising administering to an individual in need thereof a therapeutic combination of claim 1 or claim 2, or a pharmaceutical composition or formulation, or a probiotic composition of claim 3 or claim 4, wherein optionally the individual is a mammal, a human, or an animal, optionally a cattle or sheep.

7. The use of claim 5, or the method of claim 6, further comprising administration of a fecal matter transplant (FMT) composition.

8. The therapeutic combination of claim 1 or 2 or the pharmaceutical composition or formulation of claim 3 or 4, wherein one or more of said organisms is live.

9. The therapeutic combination of claim 1 or 2 or the pharmaceutical composition or formulation of claim 3 or 4, wherein one or more of said organisms is inactivated or dead.

10. A method for the treatment, prevention, reversal of, or amelioration of: ulcerative colitis, Crohn's disease, collagenous colitis, microscopic colitis, lymphocytic colitis, pseudomembranous colitis, Clostridium difficile infection, diarrhoea or diarrhoea caused by Clostridium difficile infections, acute infective agents such as Salmonella, Shigella, Campylobacter, Aeromonas, Cholera and other acute gastrointestinal infections, infections which have an intracellular component, sarcoidosis, cardiac sarcoidosis, asthma, chronic H. pylori infection, irritable bowel syndrome, Type I and type II diabetes, psoriasis, multiple sclerosis (MS), obesity, infections of the lungs, cystic fibrosis, and/or Segnihparus (including S rugosus and rotundus) lung infections, the method comprising:
administering to an individual in need thereof a therapeutic combination of claim 1 or claim 2, or a pharmaceutical composition or formulation or probiotic composition of claim 3 or claim 4, for a period of time sufficient to obtain a desired therapeutic effect;
(ii) administering to said individual, after (i), a composition having anti-MAP activity, optionally wherein said composition comprises anti-MAP antibiotics, for a period of time sufficient to obtain a desired therapeutic effect;
(iii) administering to said individual, after (ii), full spectrum fecal microbiota (FSM) implant, or one or more cultured organisms found in the human gut microbiota, sufficient to obtain a desired therapeutic effect.

11. The method according to claim 10, wherein the combination or composition described in (i) comprises at least one Dietzia sp., optionally a specie as set forth in Table 1.

12. The method according to claim 10 or 11, wherein the composition or formulation of (i) comprises single or combined strains.

13. The method according to any one of claims 10 to 12, wherein the composition or formulation of (i) comprises one or more strains alive and culturable.

14. The method according to any one of claims 10 to 13, wherein the composition or formulation of (i) comprises one or more killed strains.

15. The method according to any one of claims 10 to 14, wherein the composition of (ii) comprises one or more antibiotics selected from the group consisting of rifabutin, clofazimine, clarithromycin, metranidazole, ethambutol or mixtures of any thereof.

16. The method according to any one of claims 10 to 15, wherein each of steps (i), (ii), and (iii) is for a period of time, each independently selected, of between one and twelve weeks.

17. The method according to any one of claims 10 to 16, wherein in any one or more of (i), (ii), and (iii), the individual is administered the respective combination, composition, or implant on multiple occasions.

18. The method according to any one of claims 10 to 17, wherein the method comprises multiple cycles of (i), (ii), and (iii).

19. The method according to any one of claims 10 to 18, wherein administration of FSM is via colonoscopy, naso-gastric or naso-jejunal tube, or via enema.

20. The method according to any one of claims 10 to 19, wherein the individual is a human.

21. The method according to any one of claims 10 to 16, wherein the disease is Crohn's disease, colitis, indeterminate colitis, sarcoidosis, microscopic or collagenous colitis.
